(19) United States
(12) Patent Application Publication
(10) Pub. No.: US 2024/0318220 A1
(43) Pub. Date: Sep. 26, 2024

(54) IONIC LIQUIDS BASED ON UNNATURAL AMINO ACIDS, PREPARATION METHODS THEREOF, AND APPLICATIONS THEREOF

(71) Applicant: PEKING UNIVERSITY, Beijing (CN)

(72) Inventors: Qing XIA, Beijing (CN); Yujie SHI, Beijing (CN); Ningning SHI, Beijing (CN)

(21) Appl. No.: 18/680,271

(22) Filed: May 31, 2024

Related U.S. Application Data
(63) Continuation of application No. PCT/CN2023/111283, filed on Aug. 4, 2023.

(30) Foreign Application Priority Data
Aug. 29, 2022 (CN) 202211042820.8

(51) Int. Cl. C12P 21/00; C12N 9/00

(52) U.S. Cl. CPC C12P 21/00; C12N 9/93; C12Y 601/01001; C12Y 601/01004; C12Y 601/01026

(57) ABSTRACT

This invention relates to an unnatural amino acid-based ionic liquid, and preparation methods and applications thereof. It specifically provides a combination for preparing a protein comprising an unnatural amino acid, comprising: (1) one or more aminoacyl-tRNA synthetases capable of binding to a mutated tRNA; (2) one or more mutated tRNAs with an anti-codon loop mutated to complement a termination codon; (3) various unnatural amino acid-based ionic liquids. The combination can be used for recombinant expression of a target protein comprising an unnatural amino acid. The unnatural amino acid-based ionic liquids can improve the read-through efficiency of the genetic codon expansion system for a premature termination codon (PTC) and/or the incorporation efficiency of unnatural amino acids.

### TECHNICAL FIELD

The present disclosure belongs to the field of biopharmaceuticals, and enhances, by synthesizing a novel unnatural amino acid-choline type ionic liquid, the solubility, incorporation efficiency, and in vivo bioavailability of insoluble unnatural amino acids, and represents a significant breakthrough for the improvement of genetic codon expansion technology and its application in disease treatment.

### BACKGROUND

In nature, there exist a total of 64 triplet codons. In most organisms, 61 of these codons are responsible for encoding the 20 natural amino acids, while the remaining three codons (UAA, UGA and UAG) do not encode any amino acids in most organisms. During translation by ribosomes, normal termination factors terminate protein synthesis.

In 2002, Krzycki's team at Ohio State University published two research papers in the journal Science, discovering that in certain archaea, Methanosarcina barkeri, the UAG codon could encode pyrrolysine, the 22^{nd} amino acid in nature. The UAG codon, located in the genome of methyltransferase, is read through by ribosomes instead of serving as a termination signal. This discovery laid a solid foundation for subsequent research on genetic codon expansion technology. Scientists led by Peter Schultz attempted to transfer the aminoacyl-tRNA synthetase/tRNA pair from a naturally occurring archaea into E. coli and found that it could encode pyrrolysine normally in bacteria, but did not encode other types of amino acids and did not affect the normal coding of other amino acids, demonstrating that this aminoacyl-tRNA synthetase and tRNA form an orthogonal pair. Further attempts to extend this orthogonal system to yeast and mammalian cells showed that it could utilize the UAG codon to encode pyrrolysine in eukaryotic organisms. Thus, it is validated that this orthogonal system can be successfully extended to other organisms than archaea, and normally plays the coding function of the UAG codon. This corresponds to the fact that the orthogonal pair can effectively convert the UAG termination codon which does not encode amino acids into a sense codon which can encode an orthogonal unnatural amino acid, and the number of sense genetic codons in organisms is expanded from 61 to 62, enabling artificial protein design and modification with such orthogonal pair at genetic level. Consequently, this method is also referred to as genetic codon expansion technology.

Over the past decade, significant advancements have been made in research on this technology. Scientists have discovered other similar orthogonal aminoacyl-tRNA synthetase/tRNA pairs in archaea. Currently, up to 15 new orthogonal translation systems have been identified. With various systems available for incorporating unnatural amino acids such as tyrosine, pyrrolysine, and phenylalanine, the selection of unnatural amino acid structures has been greatly enriched. Moreover, an increasing number of species including E. coli, mammalian cells, yeast, and insect cells now can allow incorporation of unnatural amino acids into proteins. These developments lay a foundation for the widespread application of this technology. In terms of incorporation methods, more options have been provided from the original amber stop codon to other termination codons, quadruplet codons, rare codons, and even optimized special ribosomes. Genetic codon expansion technology has important applications in protein function regulation, disease treatment, and biological control.

Despite the rapid development and promising application prospects of genetic codon expansion technology in biopharmaceuticals, there still remain several technical bottlenecks: 1) Low solubility of some unnatural amino acids. Currently, unnatural amino acids are mainly synthesized chemically in vitro, with high costs. Additionally, some functional group-bearing unnatural amino acids are insoluble in water and can only dissolve in organic solvents, which leads to cytotoxicity and thus limits their applications. Therefore, how to optimize the structure of these unnatural amino acids to improve their water solubility and allow them to function in cells is a pressing issue. 2) Low oral bioavailability of unnatural amino acids in animals. Currently, unnatural amino acids are administered mainly through local targeted delivery or intraperitoneal injection, which is inconvenient and unsafe, and has low bioavailability. They are rapidly and completely metabolized in serum, with a small amount present in target organs and tissues, necessitating frequent injections and leading to high overall dosages and costs. Therefore, the low oral bioavailability of unnatural amino acids limits the application of genetic codon expansion technology in animal-level research. 3) The efficiency of incorporating unnatural amino acids using genetic codon expansion technology needs further improvement. During protein translation, when the ribosome reaches a premature codon, orthogonal tRNA carrying an unnatural amino acid competes with prokaryotic/eukaryotic release factors for recognizing and binding to the termination codon, resulting in that unnatural amino acids cannot be 100% encoded at the PTC position. Thus, another core issue restricting the application of this technology is the incorporation efficiency of unnatural amino acids. At present, scientists have already made significant progress in improving the structures of aminoacyl-tRNA synthetases/tRNAs, and optimizing EF-Tu, eukaryotic release factors eRF1 and other translation components to improve the incorporation efficiency of unnatural amino acids. However, no research has yet been conducted to optimize unnatural amino acid substrates for improving incorporation efficiency.

Ionic liquids are liquid molten salts formed under certain conditions through the combination of cations and anions in a precise stoichiometric ratio. Typically, ionic liquids consist of organic cations and inorganic/organic anions. Commonly used cations include quaternary ammonium, imidazolium, and pyrrolidinium ions, and anions include halide, tetrafluoroborate, and hexafluorophosphate ions. The main interaction forces among the components of ionic liquids are hydrogen bonds and van der Waals forces. Meanwhile, Ionic liquids can be tailored for different physical and chemical properties by combining different cations and anions or adjusting their ratios. Ionic liquids have evolved through three generations according to their development history: The first generation, with low viscosity and high thermal stability, is sensitive to oxygen and has strong hygroscopicity and thus needs to be synthesized and applied in inert atmosphere, which limits its applications. The second generation overcomes the drawbacks of the first generation, offering high chemical stability and being used as high-performance materials and metal ion chelators. The third generation, comprising choline, amino acids, and alkyl sulfates, is recognized as “green solvents” due to their facile synthesis, high biodegradability, low toxicity, and no need of purification. The third generation has been widely used in synthetic catalysis, drug development and delivery, novel polymer materials and other fields. However, imidazolium-based ionic liquids have limited applications in drug delivery due to their high cytotoxicity. Choline-based ionic liquids possess advantages such as natural and green raw materials, low-toxicity, and good biodegradability, and thus are widely used in the field of drug delivery, with more diversified drug types and administration routes. Previous studies have demonstrated that ionic liquids can enhance the solubility of small molecules, improve drug permeability, and promote oral absorption of both small molecule drugs and biomacromolecules, thus presenting promising prospects for broad applications.

### SUMMARY

In view of the above-mentioned limitations in the prior art, the objective of this disclosure is to modify the physicochemical parameters of unnatural amino acids, improve the read-through efficiency of the genetic codon expansion system for premature termination codons (PTCs), and optimize the incorporation efficiency of unnatural amino acids into target proteins. This disclosure provides an unnatural amino acid-based ionic liquid, and preparation method and applications thereof. With the unnatural amino acid-based ionic liquid, the read-through efficiency of the genetic codon expansion system for PTCs, and/or the incorporation efficiency of unnatural amino acids are improved.

Specifically, the present disclosure provides the technical solutions as follows:

In a first aspect, the disclosure provides a combination for preparing a protein comprising an unnatural amino acid, comprising:
(1) one or more aminoacyl-tRNA synthetases capable of binding to a mutated tRNA;
(2) one or more mutated tRNAs with an anti-codon loop mutated to complement a termination codon; and
(3) an unnatural amino acid-based ionic liquid.

Further, according to the combination for preparing the protein containing the unnatural amino acid of the present disclosure, wherein the aminoacyl-tRNA synthetase(s) in (1) is/are capable of producing aminoacyl-tRNA by binding the unnatural amino acid to the mutated tRNA(s) in (2).

Further, according to the combination for preparing the protein comprising the unnatural amino acid of the present disclosure, wherein the unnatural amino acid-based ionic liquid in (3) exhibits enhanced solubility and/or bioavailability compared to a corresponding unnatural amino acid.

Preferably, according to the combination for preparing the protein comprising the unnatural amino acid of the present disclosure, wherein the unnatural amino acid-based ionic liquid in (3) is unnatural amino acid-choline, including but not limited to NAEK-choline, Anap-choline, and pAcF-choline.

Preferably, according to the combination for preparing the protein comprising the unnatural amino acid of the present disclosure, wherein the aminoacyl-tRNA synthetase(s) in (1) is selected from MmPylRS, EcLeuRS, and EcTyrRS.

Preferably, according to the combination for preparing the protein comprising the unnatural amino acid of the present disclosure, wherein the mutated tRNA(s) in (2) is/are selected from mutated tRNA^{MmPyl}, mutated tRNA^{EcLeu}, and mutated tRNA^{EcTyr}.

Preferably, according to the combination for preparing the proteins comprising the unnatural amino acid of the present disclosure, wherein:
the aminoacyl-tRNA synthetase is MmPylRS from Methanosarcina mazei, and the mutated tRNA is tRNA^{MmPyl}_{UCA};
the aminoacyl-tRNA synthetase is EcLeuRS from Escherichia coli, and the mutated tRNA is tRNA^{EcLeu}_{CUA};
the aminoacyl-tRNA synthetase is EcTyrRS from Escherichia coli, and the mutated tRNA is tRNA^{EcTyr}_{UUA}.

In a second aspect, the disclosure provides a method for recombinant expression of a target protein comprising an unnatural amino acid, comprising incorporating the unnatural amino acid into the target protein using the combination for preparing the protein comprising the unnatural amino acid of any one of the present disclosure.

Preferably, according to the method for recombinant expression of the target protein comprising the unnatural amino acid of the present disclosure, comprising recombinantly expressing the protein comprising the unnatural amino acid in an E. coli, yeast, mammalian or insect cell as a host cell;

More preferably, the unnatural amino acid is encoded by a premature termination codon (PTC).

Preferably, according to the method for recombinant expression of the target protein comprising the unnatural amino acid of the present disclosure, comprising the steps of:
(1) modifying a host cell to express one or more aminoacyl-tRNA synthetases and one or more mutated tRNAs;
(2) preparing a recombinant host cell by introducing an expression cassette comprising a nucleic acid encoding a protein comprising an unnatural amino acid into the modified host cell from step (1); and
(3) culturing the recombinant host cell from step (2) in a medium added with an unnatural amino acid based-ionic liquid.

Optionally, the method for recombinant expression of the target protein comprising the unnatural amino acids of the present disclosure may further comprise, after step (3), the step of:
(4) culturing the recombinant host cell and isolating the target protein comprising the unnatural amino acid from the culture.

In a third aspect, the disclosure provides use of an unnatural amino acid-based ionic liquid in improving read-through efficiency of a genetic codon expansion system for a PTC and/or incorporation efficiency of an unnatural amino acid,
wherein the genetic codon expansion system comprises one or more aminoacyl-tRNA synthetases and one or more mutated tRNAs;
the unnatural amino acid-based ionic liquid comprises but is not limited to Ch-NAEK, Ch-Anap, and Ch-pAcF.

Preferably, according to the use of the unnatural amino acid-based ionic liquid in improving read-through efficiency of the genetic codon expansion system for the PTC and/or incorporation efficiency of the unnatural amino acid of the present disclosure, wherein the unnatural amino acid-based ionic liquid, in place of or partially in place of an unnatural amino acid, is added to a recombinant cell culture medium.

In a fourth aspect, the disclosure provides an unnatural amino acid-based ionic liquid, prepared by using choline and an unnatural amino acid as raw materials, wherein the molar ratio of choline to the unnatural amino acid is 1:0.1-10; preferably, the unnatural amino acid is selected from NAEK, Anap and pAcF.

The beneficial effects of this method are as follow:
1) Based on the three genetic codon expansion systems established earlier, three types of unnatural amino acids (Anap, NAEK and pAcF) can be chemically synthesized into unnatural amino acid-choline ionic liquids, which were validated to have been successfully synthesized by mass spectrometry identification, infrared spectroscopy characterization, and nuclear magnetic resonance hydrogen spectrum characterization.
2) The three types of unnatural amino acid-choline ionic liquids can be used to improve the genetic codon expansion system. The unnatural amino acid-choline ionic liquids prepared according to the present disclosure are detected to have significantly improved solubility and bioavailability, low cytotoxicity and good safety. Especially surprisingly, the unnatural amino acid-choline ionic liquids of the present disclosure also improve the read-through efficiency of the genetic codon expansion system for PTCs and the incorporation efficiency of the unnatural amino acids.
3) Pharmacokinetic and pharmacodynamic studies were conducted in a mouse model, especially in a muscular dystrophy mouse model, by using the unnatural amino acid-choline ionic liquids of the present disclosure in combination with the three genetic codon expansion systems established earlier, verifying their capability of restoration of Dystrophin expression in muscle tissues, thus laying a foundation for preclinical research in biopharmaceuticals.

### BRIEF DESCRIPTION OF THE DRAWINGS

The above-mentioned objectives, features and advantages of the exemplary embodiments of the present disclosure, as well as other objectives, features, and advantages, will become more apparent upon reference to the accompanying drawings. In the drawings, several embodiments of the disclosure are shown in an exemplary and non-limiting manner, with the same or corresponding numbers indicating the same or corresponding parts, wherein:

FIG. 1A. Synthesis Route of Three UAA-Choline (Ch-UAA) Ionic Liquids The synthesis routes of the three UAA-Choline ionic liquids are shown in FIG. 1A. The ionic liquids with unnatural amino acids as anions and choline as cations were prepared through the synthesis routes.

FIG. 1B. Appearance of Three UAA-Choline Ionic Liquids The physical appearance of the three different UAA-Choline ionic liquids is shown in FIG. 1B. Ch-NAEK appeared as a pale yellow transparent liquid at room temperature with good fluidity; Anap-choline (Ch-Anap) appeared as liquid at room temperature and was brown in color (possibly due to a higher proportion of choline); pAcF-choline (Ch-pAcF) was viscous and brownish yellow at room temperature and quickly melt into a liquid at 50 C°.

FIG. 2. Solubility Improvement Results of UAA by the Three UAA-Choline Type Ionic Liquids The solubility of the three UAA-choline type ionic liquids in water was significantly higher than that of the corresponding free unnatural amino acid powders. The solubility of Ch-NAEK in water exceeded 70%. The corresponding UAAs were dissolved in water and measured for solubility.

FIG. 3. Safety Concentration Screening of the Three UAA-Choline Type Ionic Liquids in 293T Cells The number of cells started to decline as the concentration of added Ch-NAEK/pAcF exceeded 2 mM, and then decreased rapidly with the increase of the concentration of the Ch-UAAs, indicating that 2 mM is the maximum safe concentration for the above two Ch-UAAs at the cellular level. Similarly, the maximum safe concentration for Ch-Anap was 500 μM.

FIG. 4A. Fluorescence Image Showing UAA Incorporation Efficiency in Recombinant Expression of GFP in 293T Cells using the Three UAA-Choline Type Ionic Liquids FIG. 4A shows that the three UAA-Choline type ionic liquids in the culture medium could normally participate in the protein translation process, allowing read-through of the termination codon TAA.

FIG. 4B. Flow Cytometry Analysis Graph Showing UAA Incorporation Efficiency in Recombinant Expression of GFP in 293T Cells using the Three UAA-Choline Type Ionic Liquids FIG. 4B shows that the read-through efficiency of TAA in the three UAA-Choline type ionic liquid groups was higher than that of the UAA aqueous solution group. The read-through efficiency in the Ch-NAEK group was improved by about 20%, which is the most significant; the read-through efficiency in the Ch-Anap group was improved by over 5%; and the read-through efficiency in the Ch-pAcF group was improved by about 10%.

FIG. 5A. Immunoblotting Result Showing Improved Bioavailability of UAA in Mice with the Three UAA-Choline Type Ionic Liquids FIG. 5A shows that the restored expression of the GFP protein in muscle tissue was comparable between pylRS-tRNA-GFP^{39TAA} transgenic mice orally dosed with 30 mg and 50 mg Ch-NAEK. Therefore, the dose of 30 mg Ch-NAEK was chosen as the preferred oral dose.

FIG. 5B. Serum Concentration Curve of NAEK in Mice After Oral Administration of Ch-NAEK FIG. 5B shows that the bioavailability of NAEK in mice in the Ch-NAEK group was higher than that in the NAEK-aqueous solution group. The NAEK content in the serum reached a maximum of about 1.6 μg/mL two hours after oral administration, as evidenced by the curve in the NAEK-aqueous solution group. Subsequently, 8 hours after oral administration, the NAEK content in the serum diminished to nearly zero, indicating rapid metabolism of NAEK, which could be completed in 6 hours. The curve in the Ch-NAEK group revealed that 9 hours after oral administration, the serum NAEK concentration reached a peak of approximately 7 μg/mL, which was about 7 times that of the control group. Furthermore, only 22 hours after oral administration, the serum NAEK content approached zero, indicating complete metabolism within approximately 15 hours. These results demonstrate that the Ch-NAEK formulation significantly increases the utilization rate of NAEK in mice, prolongs its half-life, enhances the absorption rate of NAEK by mouse cells, and extends its metabolic duration, laying a foundation for improving the utilization rate of NAEK in different tissues.

FIG. 6. Tissue Distribution Determination of the UAA-Choline Type Ionic Liquids in Mice The NAEK content in various tissues of mice in the Ch-NAEK group was higher than that in the NAEK-aqueous solution group, with a more pronounced increase of NAEK content observed in muscle, stomach, brain and heart tissues.

FIG. 7. Detection of Read-Through Expression of Fluorescent Protein in Different Tissues of Mice by Immunoblotting The GFP read-through expression in heart, brain, stomach, and muscle tissues of mice in the Ch-NAEK group was significantly higher than that in the NAEK-aqueous solution group, with a most pronounced increase of GFP expression observed in stomach tissue. These results indicate that the increased NAEK content in tissues contributes to an enhanced rate of GFP protein read-through, which is consistent with the trend of NAEK content in these aforementioned tissues.

FIG. 8. Detection of Full-Length Expression of Dystrophin in Mouse Muscle Tissue by Immunoblotting mdx mice showed significant restoration of full-length expression of the disease protein after 2 weeks of oral administration of the Ch-NAEK solution, while the restoration of the disease protein expression in the NAEK aqueous solution group was very low. Furthermore, there was no significant difference in restoring protein expression between the 2-week and 4-week oral groups, indicating that after 2 weeks of oral administration, the restoration of the disease protein expression in mice has reached a plateau, at approximately 45%. This level of expression was significantly higher than that in both the NAEK aqueous solution group and the previous intraperitoneal injection group observed at the same time point. These results suggest that Ch-NAEK can effectively increase the utilization rate of NAEK in mice, achieving efficient expression of the disease protein with only a low dose of NAEK.

FIG. 9A. Identification of Ch-NAEK (Target Molecular Peak: 466) by Mass Spectrometry

FIG. 9B. Identification of Ch-pAcF (Target Molecular Peak: 414) by Mass Spectrometry

FIG. 9C. Identification of Ch-Anap (Target Molecular Peak: 895) by Mass Spectrometry The mass peaks of the three synthesized UAA-Choline type ionic liquids identified by mass spectrometry corresponded to the theoretical values, indicating high purity of the synthesized products.

FIG. 10A. Identification of Ch-NAEK by Nuclear Magnetic Resonance Hydrogen Spectrum

FIG. 10B. Identification of Ch-pAcF by Nuclear Magnetic Resonance Hydrogen Spectrum

FIG. 10C. Identification of Ch-Anap by Nuclear Magnetic Resonance Hydrogen Spectrum By analyzing the nuclear magnetic resonance hydrogen spectrum of the three groups, Anap/Ch-Anap, NAEK/Ch-NAEK, and pAcF/Ch-pAcF, it can be known that the three newly synthesized Ch-UAAs were successfully synthesized.

FIG. 11A. Infrared Spectrum of Ch-NAEK

FIG. 11B. Infrared Spectrum of Ch-pAcF

FIG. 11C. Infrared Spectrum of Ch-Anap The infrared spectrum peak shapes of Ch-UAA and UAA were basically consistent, with the peak shapes of key functional groups being consistent with each other. Due to the influence of choline ions, the basic positions of some peaks shifted.

FIG. 12. Flow Diagram of Treating DMD Disease in Mdx Mouse Model Using Ionic Liquid Ch-NAEK and the Codon Expansion System Experimental exploration was conducted to investigate the treatment of DMD disease. Following successful synthesis, identification and activity evaluation of the three ionic liquids Ch-NAEK, Ch-pAcF and Ch-Anap, Ch-NAEK was selected as the preferred ionic liquid for subsequent disease treatment. mdx mice with DMD disease were initially intramuscularly injected with AAV-MmpylRS-tRNA virus and subsequently orally administered with Ch-NAEK (at a mass volume fraction of 70%) daily for four weeks. Muscle tissue samples were collected in the first, second, and fourth weeks for detection of Dystrophin protein restoration. HE pathological staining of mouse muscle tissue, determination of serum CK kinase content, and testing of mouse grip strength were performed. The therapeutic efficacy of the delivery method was comprehensively evaluated in a mouse model with disease, showing that this formulation significantly improved the delivery and incorporation efficiency of unnatural amino acids, which enables oral administration of unnatural amino acids for DMD disease treatment, and represents a significant breakthrough in the treatment of nonsense mutation diseases.

### DETAILED DESCRIPTION

To make the objectives, technical solutions, and advantages of this disclosure clearer, further detailed descriptions of the disclosure are provided in conjunction with the drawings.

### Example 1: Synthesis of Three UAA-Choline Type Ionic Liquids by Chemical Synthesis

### 1. Synthesis Route and Method of Ch-NAEK

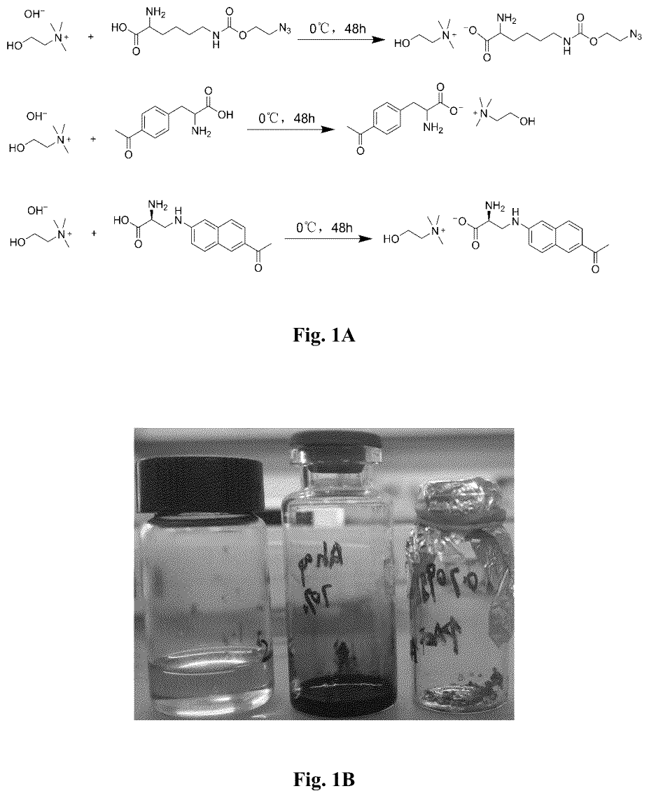

The specific synthesis route is shown in FIG. 1A, with the specific process as follows: the Ch-NAEK ionic liquid was synthesized by using choline hydroxide and NAEK as reaction materials in a ratio of choline hydroxide:NAEK=1:1. Firstly, an appropriate amount of NAEK was weighed and placed in a reaction flask, added with an appropriate amount of water, and stirred with a stirrer to allow NAEK to dissolve in water; then choline hydroxide containing 45% methanol was evaporated with a rotary evaporator to remove methanol, and an appropriate amount of choline hydroxide was weighed and added to the reaction flask with a glass dropper, maintaining a temperature of 0 C° for 48 hours. After the reaction, the product was rotated using a rotary evaporator at 55 C° to remove water, added with the amino acid precipitant ACN/MeOH (9/1), followed by filtration. The filtrate was collected, and then rotated at 40 C° to remove the organic solvent. The final liquid substance was collected after drying in a 60 C°-vacuum oven for 48 h.

### 2. Synthesis Method of Anap-Choline

The method was the same as above, with choline:Anap=6:1.

3. Synthesis Method of pAcF-Choline

The method was the same as above, with choline:pAcF=1:1.

The appearance of the three synthesized ionic liquids Ch-NAEK, Ch-Anap, and Ch-pAcF is shown in FIG. 1B.

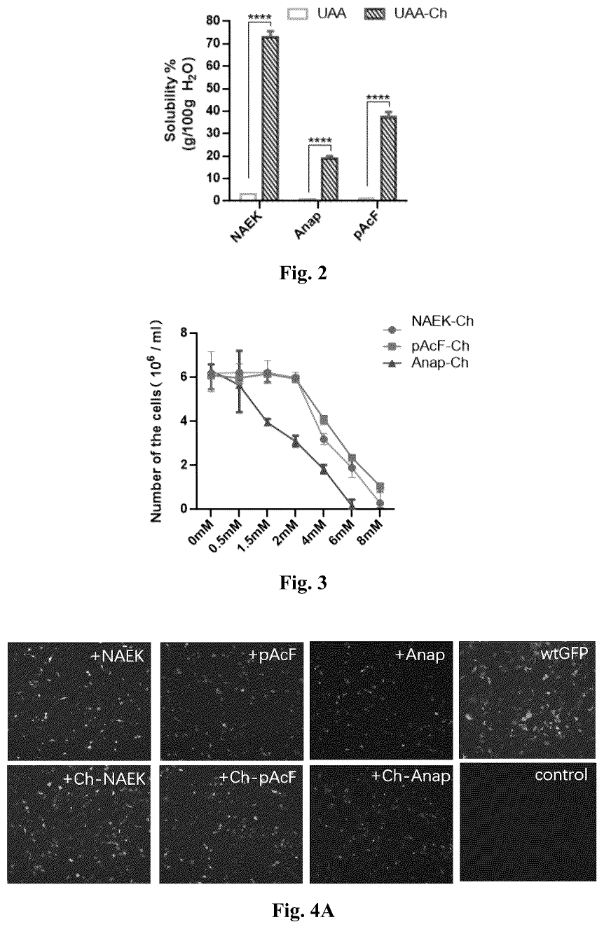

The solubility of the three ionic liquids in H₂O was tested, with results shown in FIG. 2.

### Example 2: Identification and Characterization of Three UAA-Choline Type Ionic Liquids

### 1. Infrared Spectroscopy Identification

1) Sample preparation: 20 mg of each of the three UAA powders, NAEK, Anap, and pAcF, and the corresponding molar amounts of the UAA-Choline type ionic liquids were taken and placed in 1.5 mL centrifuge tubes, respectively, and kept dry.
2) Sample testing: The six samples were added to the sample inlet of an infrared spectrometer for instrumental analysis. The resolution was 4 cm⁻¹, and the scanning range was 4,200-400 cm⁻¹ (FIG. 1A-C).

### 2. Nuclear Magnetic Resonance Hydrogen Spectrum Identification

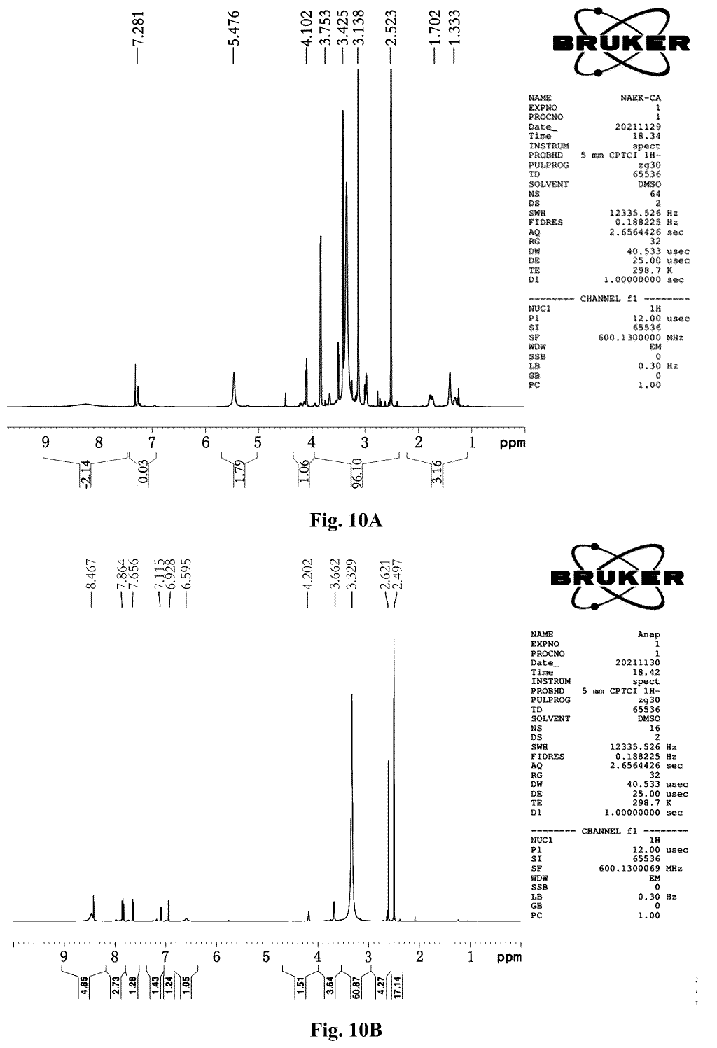

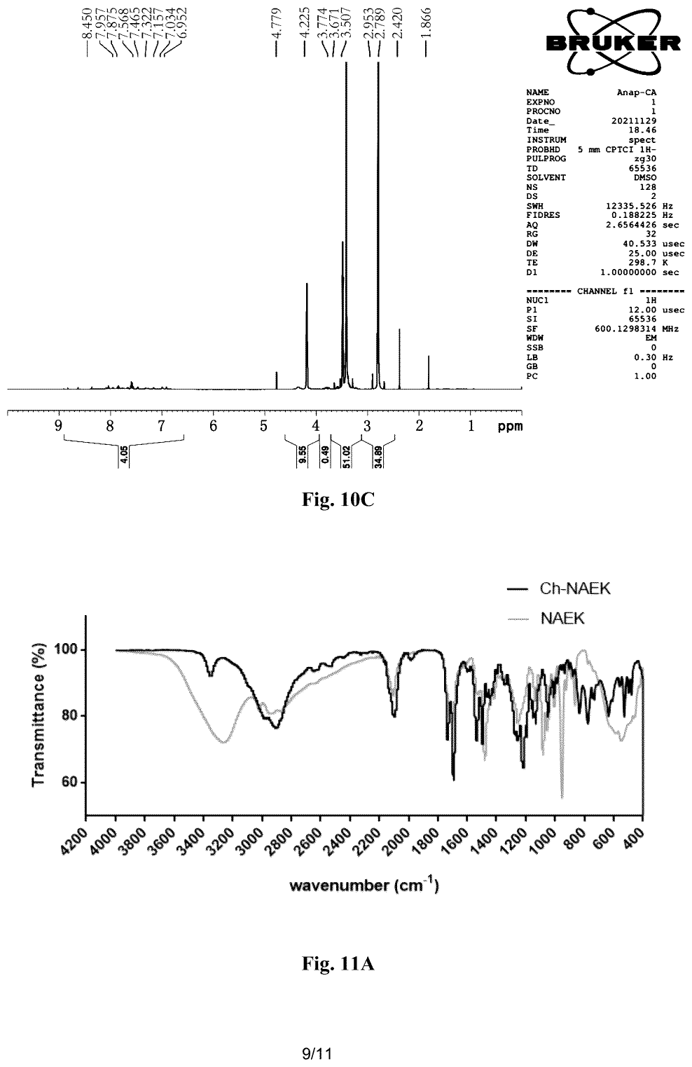

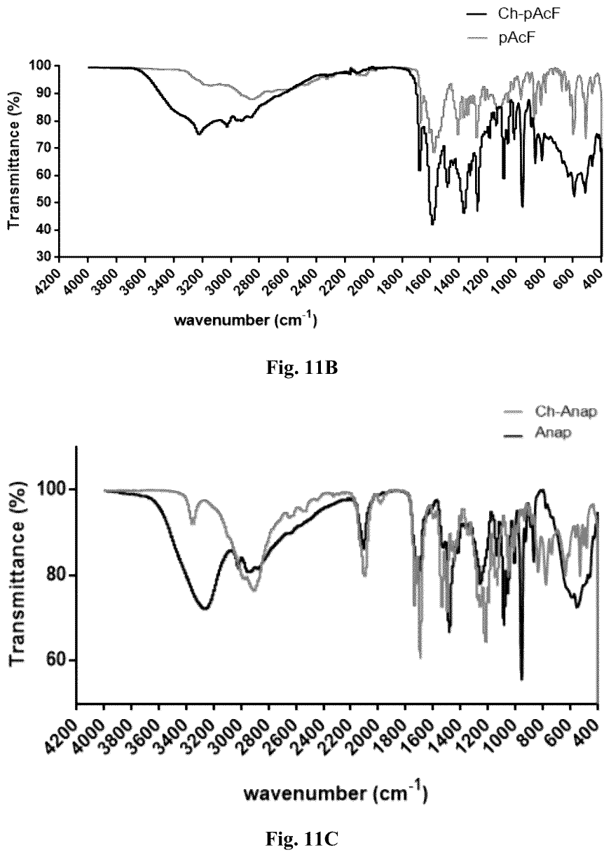

20 mg of the ionic liquids were placed in a 3 mL centrifuge tube, fully dissolved in deuterated DMSO, transferred to a nuclear magnetic tube, labeled, and then sent for testing (FIG. 10A-C).

### 3. Mass Spectrometry Identification

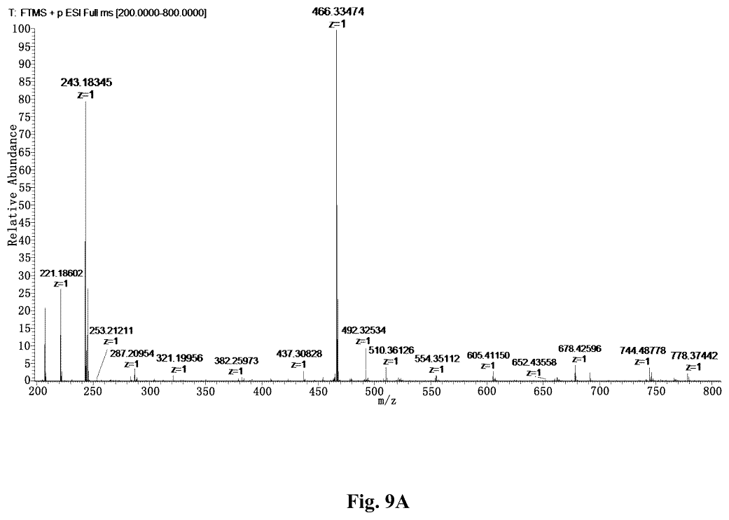

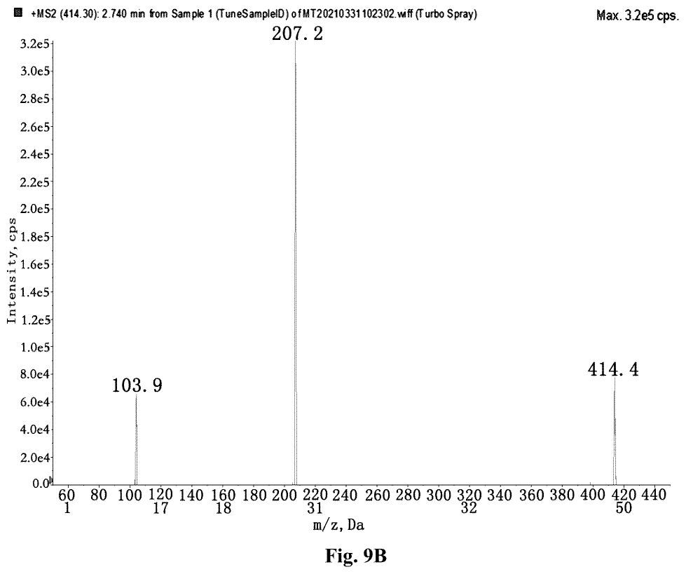

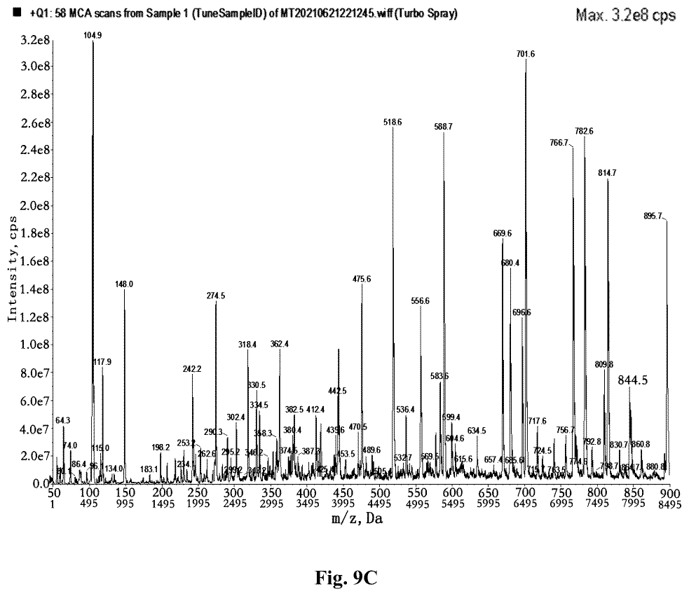

The synthesized three unnatural amino acid-choline compounds were dissolved in a small amount of deionized water, made into a 1 μg/ml solution, and loaded for total molecular weight mass spectrometry analysis (FIG. 9A-C).

### 4. Data Processing and Analysis

As the number of choline molecules differs in the three newly synthesized substances, choline ions may dissociate in solution. Therefore, each substance may exist in multiple forms. According to the requirements of mass spectrometry identification, nuclear magnetic resonance hydrogen spectrum interpretation, and infrared spectrum interpretation, data analysis was conducted to verify the accuracy of the synthesized compounds. The specific mass spectrometry data was as follows:

$$Ch - NAEK : 259 + 104 \times 2 = 467 ( 1 NAEK ion + 2 choline ions )$$

$$Ch - pAcF : 310 + 104 = 414 ( 1 pAcF ion + 1 choline ion )$$

$$Ch - Anap : 273 + 104 \times 6 - 2 = 895 ( 1 Anap ion + 6 choline ions ) .$$

### Example 3: Toxicity and Safety of UAA-Choline Type Ionic Liquids in Mammalian Cells

### 1. Preparation of Concentration Gradients of the Three UAA-Choline Type Ionic Liquids

Firstly, an appropriate amount of the UAA-choline ionic liquids were weighed and dissolved in water to prepare three UAA-Choline (Ch-NAEK, Ch-Anap, and Ch-pAcF) solutions with a concentration of 50 mM UAA-Choline. Subsequently, these solutions were diluted with 293T cell culture media to obtain 293T cell culture media containing the UAA-Choline type ionic liquids at concentrations of 0 mM, 0.5 mM, 1.5 mM, 2 mM, 4 mM, 6 mM, and 8 mM for future use.

### 2. Cell Viability Test

a. Cell plating: Cells in a 10 cm dish were digested into single cells by adding 1 mL of 0.25% Trypsin-EDTA digestive solution, and counted using a cell counter after resuspension. The plating density for a six-well plate was 3×10⁵ cells per well. After thorough mixing, the cells were transferred to a 37 C° incubator and cultured overnight until the cell number reached about 70%, followed by transfection.
b. Cell number versus UAA-Choline concentration curve: The 293T cells were added with cell culture media containing different concentrations of UAA-Choline, observed for 48 h and tested for viability. After 48 h of culture, the cells were digested and collected, and the cell number was counted using a cell counter to create a curve fitting the cell number and the UAA-Choline concentrations.
c. Cell growth curve: Two experimental groups were set up, with two replicates in each group, one with an appropriate amount of UAA aqueous solution added to plated 293T cells, and the other with an equimolar amount of UAA-Choline type ionic liquid added to the cells. Cells from each group were cultured, digested, collected and counted at time points including 0 h, 6 h, 24 h, 48 h, 56 h, and 72 h, to plot cell growth curves under different conditions (FIG. 3).

### Example 4: Enhancement of UAA Incorporation Efficiency in Animal Cells Using UAA-Choline Ionic Liquids

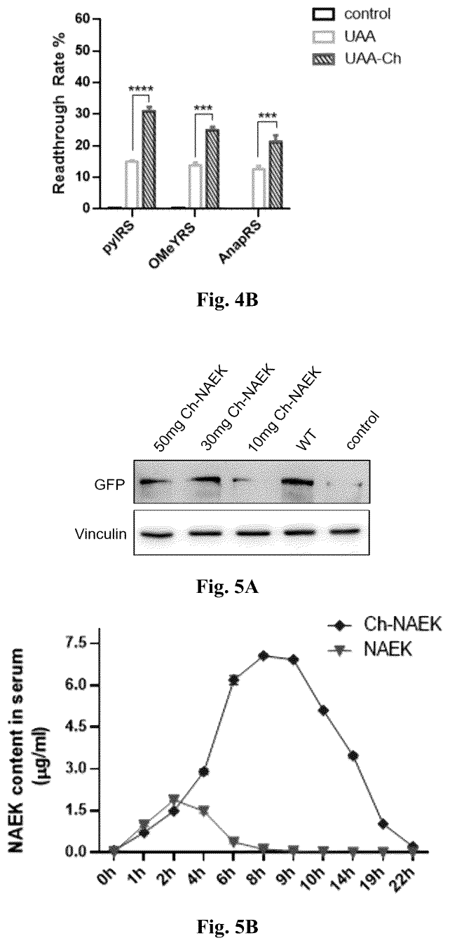

The UAA incorporation efficiency in animal cells using the UAA-Choline ionic liquids was tested by referring to the method in patent application No. 202111050643.3. In brief, three types of aminoacyl synthetases and mutant tRNA orthogonal codon expansion systems were transfected into 293T cells, followed by a plasmid carrying the GFP recombinant expression cassette with a PTC. Six hours after plasmid transfection, the medium was changed. Cell culture media containing 1 mM/100 μM UAA-Choline and 1 mM/100 μM UAA were prepared and added to the 293T cells. After 48 hours of cultivation, the cells were observed and photographed under a fluorescence microscope (FIG. 4A), and analyzed using flow cytometry (FIG. 4B), to compare the read-through efficiency of TAA in three genetic codon expansion systems under the addition of two different formulations of unnatural amino acids.

### Example 5: Optimization of Oral Dosage of Ch-NAEK in Mice

### 1. Determination of the Optimal Oral Safety Concentration of Ch-NAEK

a. Firstly, Ch-NAEK solutions were prepared at mass-volume fractions of 100%, 90%, 80%, 70%, 60%, and 50%.
b. Mice were orally administered with different mass-volume fractions of the Ch-NAEK solutions, and observed for their growth status.

### 2. Determination of the Optimal Oral Dosage

In earlier stages, transgenic mice expressing pylRS-tRNA-GFP^{39TAA} were successfully prepared by prokaryotic microinjection. Oral dosage groups of 10 mg, 30 mg, and 50 mg of Ch-NAEK per day were set. pylRS-tRNA-GFP^{39TAA} transgenic mice were orally administered with three different doses of Ch-NAEK, respectively. One week later, muscle tissue of mice from each group was extracted to detect the restored expression of GFP protein (FIG. 5A).

### Example 6: Detection of In Vivo Bioavailability of Ch-NAEK in Mice

Two experimental groups were set, with 12 mice in each group. In one group, each mouse was orally administered with 30 mg NAEK aqueous solution, and in the other group, each mouse was orally administered with an equimolar amount of Ch-NAEK. Blood was collected from the orbital plexus of the mice at 1 h, 2 h, 4 h, 6 h, 8 h, 9 h, 10 h, 14 h, 19 h, and 22 h time points to plot the NAEK serum concentration in mice as function of time (FIG. 5B).

### Example 7: Determination of UAA Content in Different Tissues in Mice

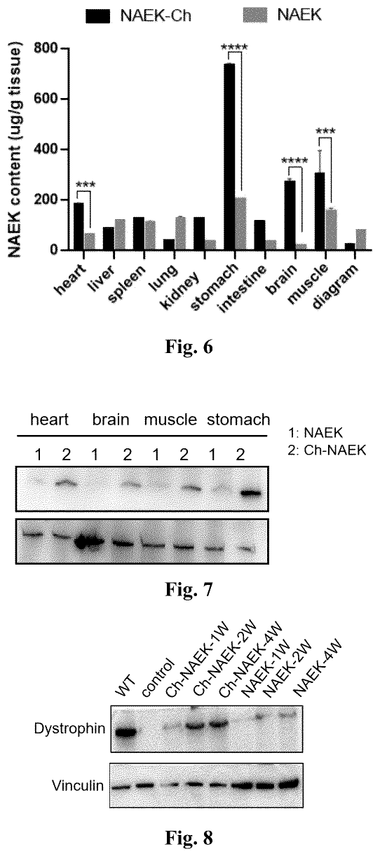

Mice in two experimental groups were orally administered with equimolar amounts of NAEK, and euthanized after 9 hours to harvest organs and tissues. The organs and tissues were placed in a homogenizer tube and homogenized in deionized water at a concentration of 0.1 g/mL. A bar graph of NAEK content in different tissues 9 hours after oral administration of different formulations of NAEK was plotted (FIG. 6).

### Example 8: Determination of GFP Protein Expression in Different Tissues in Mice

pylRS-tRNA-GFP^{39TAA} transgenic mice in two experimental groups were orally administered with equimolar amounts of NAEK daily. After one week, heart, muscle, brain, and liver tissues were harvested, homogenized, and subjected to proteins extraction for immunoblotting (FIG. 7).

### Example 9: Optimization of Full-Length Expression of Dystrophin in Mdx Mice with UAA-Choline Type Ionic Liquids

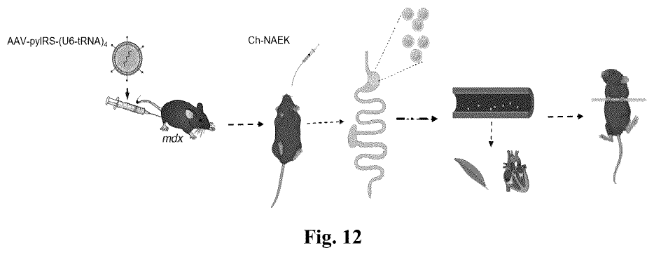

mdx mice, a classic mouse model for DMD (Duchenne Muscular Dystrophy) research, have a nonsense mutation of TAA at the 23^{rd} codon of Dystrophin exon, resulting in the failure of normal expression of Dystrophin, and thus leading to symptoms of muscle atrophy in muscle and heart tissues in mice. mdx mice injected with AAV-MmpylRS-tRNA virus were divided into two experimental groups. One group of mice was orally administered with NAEK solution, while the other group of mice was orally administered with an equimolar amount of Ch-NAEK solution. The therapeutic effects were evaluated at the time points of 1, 2, and 4 weeks after administration. Anterior tibial muscle tissues from control and experimental groups of mdx mice were harvested and subjected to tissue processing and protein extraction for immunoblotting at the time points of 1, 2, and 4 weeks after oral NAEK administration (FIG. 8, FIG. 12).

These aforementioned examples represent preferred embodiments of this disclosure, which are only intended to clarify and facilitate the understanding of the spirit of this disclosure, but not to limit the disclosure. Any modifications, replacements, and improvements made within the spirit and principles of this disclosure should be included in the protection scope of the appended claims of this disclosure.

1. A combination for preparing a protein comprising an unnatural amino acid, comprising:
(1) one or more aminoacyl-tRNA synthetases capable of binding to a mutated tRNA;
(2) one or more mutated tRNAs with an anti-codon loop mutated to complement a termination codon; and
(3) an unnatural amino acid-based ionic liquid, which is an unnatural amino acid-choline ionic liquid, produced through chemical synthesis using choline and the unnatural amino acid as raw materials;
wherein the aminoacyl-tRNA synthetase(s) in (1) is/are capable of producing aminoacyl-tRNA by binding the unnatural amino acid to the mutated tRNA(s) in (2); and
the unnatural amino acid-based ionic liquid in (3) exhibits enhanced solubility and/or bioavailability compared to the unnatural amino acid;
the aminoacyl-tRNA synthetase is MmPylRs from Methanosarcina mazei, and the mutated tRNA is tRNA^{MmPyl}_{UCA};
the aminoacyl-tRNA synthetase is EcLeuRs from Escherichia coli, and the mutated tRNA is tRNA^{EcLeu}_{CUA};
the aminoacyl-tRNA synthetase is EcTyrRs from Escherichia coli, and the mutated tRNA is tRNA^{EcTyr}_{UUA}.

2. The combination for preparing the protein comprising the unnatural amino acid according to claim 1, wherein the unnatural amino acid-choline ionic liquid is Ch-NAEK, Ch-pAcF, Ch-Anap.

3. A method for recombinant expression of a target protein comprising an unnatural amino acid, comprising incorporating the unnatural amino acid into the target protein using the combination for preparing the protein comprising the unnatural amino acid of claim 1.

4. The method according to claim 3, comprising recombinantly expressing the target protein comprising the unnatural amino acid in an E. coli, yeast, mammalian or insect cell as a host cell, wherein the unnatural amino acid is encoded by a premature termination codon (PTC).

5. The method according to claim 4, comprising the steps of:
(1) modifying a host cell to express one or more aminoacyl-tRNA synthetases and one or more mutated tRNAs;
(2) preparing a recombinant host cell by introducing an expression cassette comprising a nucleic acid encoding a protein comprising an unnatural amino acid into the modified host cells from step (1); and
(3) culturing the recombinant host cell from step 2 in a medium added with an unnatural amino acid based-ionic liquid.

6. The method according to claim 5, further comprising, after step (3), the step of:
(4) culturing the recombinant host cell and isolating the target protein comprising the unnatural amino acid from the culture.

7-9. (canceled)
